(19) United States
(12) Patent Application Publication
(10) Pub. No.: US 2025/0366938 A1
(43) Pub. Date: Dec. 4, 2025

(54) METHOD AND APPARATUS FOR CONTROLLING THE MOVEMENT OF AN ELONGATED MEDICAL INSTRUMENT WITHIN A BODY

(71) Applicant: Siemens Healthineers AG, Forchheim (DE)

(72) Inventor: Marcus Pfister, Bubenreuth (DE)

(21) Appl. No.: 19/220,067

(22) Filed: May 27, 2025

(30) Foreign Application Priority Data
May 28, 2024 (DE) 10 2024 204 912.3

(51) Int. Cl. A61B 34/30; A61B 34/00; A61B 34/10; A61B 34/20; A61B 90/00; G16H 40/63

(52) U.S. Cl. CPC A61B 34/30; A61B 34/10; A61B 34/20; A61B 34/25; A61B 90/06; G16H 40/63; A61B 2034/102; A61B 2034/2046; A61B 2034/301; A61B 2090/064

(57) ABSTRACT

A method for controlling movement of an elongated medical instrument within a body that along its length includes an optical fiber configured for optical shape capture is provided. An insertion section of the fiber is located in the body. The method includes measuring a bend at multiple positions of the fiber, at least along the insertion section of the fiber. A three-dimensional (3D) shape of the insertion section of the fiber in the body is determined from the measured bend. A sequence of movements, with which a holding section of the instrument is to be moved, is determined based on the shape of the insertion section, so that the bend of the instrument in the insertion section may be decreased if the holding section is moved correspondingly. The sequence of movements is output.

This application claims the benefit of German Patent Application No. DE 10 2024 204 912.3, filed on May 28, 2024, which is hereby incorporated by reference in its entirety.

### BACKGROUND

The present embodiments relate to controlling movement of an elongated medical instrument within a body.

In minimally invasive examinations or procedures (e.g., supported by C-arm angiography systems), treatments and/or diagnoses are performed with the help of instruments that are inserted into the body via small incisions (e.g., in the groin). For navigation to the region of interest (e.g., a vascular target) or for visualization of catheters and other instruments, X-ray radioscopy (e.g., fluoroscopy) has generally been employed to date.

To minimize the disadvantage of the radiation, more recent methods use 3D fiber optics that may identify the shape and position of an inserted optical fiber using intrinsic light reflections. This shape recognition may take place in isolation or together with imaging (e.g., visualization by a C-arm angiography system). Thus, for example, guide wires in a body may be visualized by measuring their shape and position and displaying the guide wires virtually in a model or image recording of the body. For example, treatment of aortic aneurysms by insertion of a stent graft is a specific therapy in which these instruments are useful. For this purpose, the instrument inserted (e.g., a guidewire or a catheter) has an optical fiber along its length for localization, which is firmly connected to the instrument.

Since the tip of an instrument may be far away from the “manipulation point” (e.g., mostly the groin area), a major problem with such procedures is that complications may arise as a result of the inserted instrument “winding up” or “coiling up” at its tip, primarily due to the energy stored in the instrument. These convolutions may store kinetic energy via the elasticity of the instrument, which may damage the body (e.g., its vascular system) if it is released by a sudden unwinding. For example, in robotic procedures with a lack of haptic feedback, this may represent a serious problem.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a method and an apparatus for controlling movement of an elongated medical instrument within a body, with which the disadvantages described above may be prevented, are provided.

A method of the present embodiments serves to control movement of an elongated medical instrument within a body, which, along its length, includes an optical fiber configured for optical shape capture. An insertion section of the fiber is located in the body. The method includes the following acts: measuring a bend at multiple positions of the fiber, at least along the insertion section of the fiber; determining a three-dimensional shape of the insertion section of the fiber in the body from the measured bend and, for example, determining positions and directions of bending forces prevailing there; determining a sequence of movements based on the shape of the insertion section and, for example, of the bending forces prevailing there, with which a holding section of the instrument is to be moved, so that the bend in the instrument in the insertion section may be decreased if the holding section is moved correspondingly; and outputting the sequence of movements.

The method relates to the control of the movement of a medical instrument within a body. In this case, the medical instrument is elongated in shape and is configured to be slid into the body. The instrument may be an endoscope, a catheter, or a guide wire or tube. Such instruments are sufficiently known in the prior art and are used to examine the body or to perform minimally invasive medical procedures within the body. The body may, in this case, be a human body, an animal body, or the body of an object (e.g., inanimate object).

The instrument has the optical fiber along its length. This fiber may, for example, be attached to a guide wire or tube or may be attached on the outside or inside to the wall of a catheter or endoscope. The optical fiber is in this case to be configured for optical shape capture. As stated above, instruments with such fibers are already the prior art.

The fiber may have a cross-sectional area of less than 5 square millimeters (e.g., less than 1 square millimeter) and a length of more than 10 cm (e.g., more than 20 cm). The fiber may be a multi-core optical fiber.

When introducing the instrument into the body, the optical fiber on the instrument is slid into the body along an insertion section. This procedure, also referred to below as sliding in, is known and may be performed during minimally invasive examinations or procedures. In this case, the instrument is inserted into the body via a small opening on the body (e.g., an incision). Since the optical fiber is fastened to the instrument, the optical fiber is inserted together with the instrument. Part of the instrument (e.g., at least its holding section) remains outside the body.

After the introduction, the instrument and thus also the fiber is partly in the body. The part of the fiber that was slid into the body is referred to as the “insertion section” to better characterize the part of the fiber for the further steps. The insertion section is thus the length of the fiber that is located in the body.

While the instrument is slid into the body, the bend in the fiber is measured at multiple positions. This may be done along the entire fiber or just along the insertion section of the fiber. The term “bend” also includes the torsion (e.g., a “bend;” here basically includes a bend orthogonal and/or parallel to the longitudinal axis). The entire fiber is generally measured. However, all measurements at positions outside the body (e.g., outside the insertion section) may be ignored if desired. The measurement, which is also referred to as fiber-optic “shape sensing” or “shape capture,” is known in the prior art.

Fiber-optic shape capture is prior art and enables the curvature and shape of a fiber to be determined in 2D and 3D. A typical fiber-optic shape capture system consists of a sensor, a measuring device, and a computing unit with algorithms for evaluating the measured data. In principle, during the measurement, the strain in the fiber is determined by interference of multiple light beams that are sent through the fiber and are reflected in the fiber. In a fiber consisting of a bundle of multiple optical waveguides, some of the outer optical waveguides experience a relative stress or compression relative to the central optical waveguide and hence register positive and negative induced strain changes. To calculate a local curvature or the bending radius, the relative strains of the fibers are measured and processed. To determine the curvature profile of the fiber, the measurements are processed using special reconstruction algorithms. Not only bends about a point may be determined in this case, but also radii of curvature, directions, forces, and torsions. Thus, the three-dimensional shape of the insertion section of the fiber in the body may be determined from the measured bend.

Since the restoring torque of the fiber is known, the positions and directions of the bending forces prevailing there may also be determined. These lie at the bends and are directed to where the fiber is to move in order to achieve a straight shape.

Thus, the radii of curvature or the angles of deflection of torsions and positions of bends are now known. From this, the shape of the fiber in the body (e.g., in the insertion section) may be derived. Where the fiber is bent (e.g., also twisted), bending forces having a strength that depends on the bending radius (e.g., inversely proportionally) and having a direction that depends on an orientation of the bend exist.

The aim is to have a shape of the fiber that is as straight as possible. In this case, this shape is not necessarily absolutely straight, but may correspond to the shortest possible (e.g., harmless) route in the body (e.g., the route of a blood vessel or branch into which the instrument was slid).

A sequence of movements is now determined, with which the fiber may assume this route. This route is, for example, achieved if it was possible to decrease the bend of the instrument in the insertion section and thus also the bending forces. This route may be achieved if it was possible to minimize the bending forces of the instrument in the insertion section. In this case, the bending forces do not necessarily have to be zero, since the desired route may in fact have curves. However, it may be calculated which target bending forces the desired route should have, and the sequence of movements may be configured such that bending forces in the insertion section arise in accordance with these target bending forces. For this purpose, the fiber may, for example, be withdrawn and rotated. However, the aim may also be that in a predetermined area of the fiber (e.g., the area at its tip), no bending force exceeds a predetermined limit value or no radius of curvature falls below a predetermined limit value.

A procedure of the present embodiments is provided. First, a theoretical target route of the fiber in the body is determined (e.g., based on X-ray or CT images of the body). Then, optionally, target bending forces are calculated based on the target route. Then, an actual route of the fiber is determined, and for example, also, the bending forces on this actual route are calculated. Finally, movements that may be used for movement from the target route to the actual route are searched.

Since the holding section of the instrument is the only part with which its guidance through the body may be influenced, the sequence of movements indicates how this holding section should be moved. The sequence of movements may, for example, merely include indicators for removal and insertion of the instrument together with strains of the holding section about is longitudinal axis (e.g., rolling movement). In addition, yaw and pitch movements of the holding section may be specified.

For example, a model of the fiber or even of the instrument may be created from the information obtained. This model may be used to derive a sequence of movements. To this end, a model of the body or at least of the area of the body in which the instrument should move may also be created. Using a simulation of the movements and an experimental algorithm, the sequence of movements may then be determined. However, the sequence of movements may also be determined using theoretical calculations.

The sequence of movements is output after it is created (e.g., in the form of control commands or control instructions in text and/or image). Thus, in this case, information is output about a sequence of movements with which the holding section should be moved in order to reduce the degree and number of bends and thus to decrease the bending forces.

An apparatus of the present embodiments is used for controlling the movement of an elongated medical instrument within a body, which, along its length, includes an optical fiber configured for optical shape capture. An insertion section of the fiber is located in the body. The apparatus includes the following components: A measuring unit configured to measure a bend at multiple positions of the fiber, at least along the insertion section of the fiber; a shape determination unit configured to determine the three-dimensional shape of the insertion section in the body from the measured bend and, for example, determination of the positions and directions of the bending forces prevailing there; a movement determination unit configured to determine a sequence of movements based on the shape of the insertion section and, for example, of the bending forces prevailing there, with which a holding section of the instrument should be moved, so that the bend of the instrument in the insertion section may be decreased if the holding section is moved correspondingly; and a data interface configured to output the sequence of movements.

The function of the components of the apparatus has already been described above. The apparatus may be configured to execute a method of the present embodiments.

The present embodiments may in practice be implemented as follows: 1. First, bending forces (e.g., directions in which the fiber has been deflected or twisted) and, for example, also bending forces at each point of the fiber are determined. The shape of the instrument is also determined at the same time since the fiber is attached to the instrument.
2. From this information, a 3D model of the fiber is then created where appropriate with force vectors of the bending forces. Instead of the model (or additionally), a diagram that indicates the shape of the fiber or bending forces may also be created. Such a diagram may indicate the bending information and/or force values relative to the instrument positions.
3. The information obtained or the model or diagram are used to carry out measures to prevent or avoid complications due to instrument tensions. To this end, the sequence of movements is determined (e.g., the model is used to derive how the twisted instrument may be brought into a shape that is as tension-free as possible).
4. The sequence of movements is then performed with the holding section of the instrument in order to optimize the shape of the fiber (and thus of the instrument) or to reduce tensions. This sequence of movements may either be displayed such that a physician may perform the movements manually, or the sequence of movements may be transmitted as a command sequence so that a robot may perform the sequence of movements automatically.

The present embodiments may be implemented, for example, in the form of a computer unit with suitable software. The computer unit may, for example, have one or more microprocessors working together or the like. For example, the present embodiments may be implemented in the computer unit in the form of suitable software program parts. A largely software-based implementation has the advantage that computer units already used may easily be retrofitted by a software or firmware update in order to work in the manner of the present embodiments. In this respect, a corresponding-computer program product with a computer program that may be loaded directly into a memory facility of a computer unit, with program sections, in order to execute all acts of the method of the present embodiments if the program is executed in the computer unit may be provided. In addition to the computer program, such a computer program product may, where appropriate, include additional components such as, for example, documentation and/or additional components, including hardware components, such as for example hardware keys (e.g., dongles, etc.) for the use of the software.

A computer-readable medium (e.g., a non-transitory computer-readable storage medium), for example a memory stick, a hard disk or another portable or permanently installed data carrier, on which the program sections of the computer program which can be read and executed by a computer unit are stored, can be used for transport to the computer unit and/or for storage on or in the computer unit.

Further, embodiments and developments emerge from the following description, where one category may also be developed analogously to parts of the description to form another category, and individual features of different example embodiments or variants may also be combined to form new example embodiments or variants.

In accordance with a method, in the course of determining the three-dimensional shape of the insertion section, a three-dimensional model of the insertion section is created. Alternatively or additionally, a graph that shows the bending information along the length of the insertion section is created. In this case, the sequence of movements may be determined based on the model and/or the graph. The bending information may, for example, be used to perform a simulation, by which movements may be identified that lead to the desired result. These movements may then be assembled to form the sequence of movements.

In accordance with a form of embodiment of the method, force vectors along the length of the insertion section are calculated in the course of the determination of bending forces. As explained above, this is possible because the shape of the fiber is known and the bending forces result directly from the shape and the restoring torque of the fiber. In this case, these force vectors may be correspondingly classified into a three-dimensional model of the insertion section and/or into a graph that shows the bending information along the length of the insertion section.

To determine the sequence of movements, a shape of the fiber in a specified area may be taken into consideration (e.g., exclusively or especially) at a tip of the insertion section. Unwanted deformations of the instrument will occur first at the tip. This specified area may be maximally an area of the first 20 cm (e.g., the first 10 cm) of the fiber measured from the tip.

The bend of the instrument may often be determined while the instrument is being slid in. In this case, the three-dimensional shape of the insertion section of the fiber may be determined at intervals of time of less than one second (e.g., less than 0.1 s). Since deformations generally do not occur with a stationary instrument, the determination may be carried out (e.g., only) until the insertion of the fiber has been stopped.

The sequence of movements may include a successive sequence of movements of the holding section that specify how the holding section is to be moved so that the bending forces in the insertion section are reduced and thus also a degree or a number of bends. The movements may be configured such that at least one bending radius of the fiber increases or the number of twists is reduced.

The method may include one or more of the following acts: moving the holding section of the fiber in accordance with the sequence of movements (e.g., automatically); and/or displaying the sequence of movements for performance by a human; and/or creating a command sequence from the sequence of movements configured so that an automatic movement unit may move the holding section automatically in accordance with the sequence of movements.

The relaxation of the fiber may be started manually or automatically. A maximum total bending force may be specified, and relaxation of the fiber is started if the sum of the determined bending forces exceeds this. Alternatively, a maximum bending force may be specified, and relaxation of the fiber is started if a determined bending force at one point in the fiber exceeds this. Alternatively, a minimum bending radius may be specified, and relaxation of the fiber is started if a measured bending radius at a point in the fiber falls below this. Alternatively, a check may be made to see whether the insertion of the instrument has been stopped longer than a specified period of time, and, if so, the sequence of movements is specified. However, relaxation of the fiber or output of the sequence of movements may also be triggered by image-based methods. Relaxation of the fiber is synonymous with relaxation of the instrument, since the fiber is attached to the instrument.

In one embodiment of the method, the output of the sequence of movements or a relaxation of the instrument is started automatically if one or more of the following conditions apply: a bending radius is smaller than a specified limit value; a bending force lies above a specified limit value; the number of bends in a specified length interval of the insertion section lies above a specified limit value; the sum of the bending forces in a specified length interval of the insertion section lies above a specified limit value; parameter values of a shape of a model of the insertion section or of a shape of the insertion section shown in the image lie outside a specified value range.

In one form of embodiment of the method, the sequence of movements is determined analytically from an analysis of the determined three-dimensional shape of the insertion section and the positions and directions of the bending forces prevailing there (e.g., by analysis of a model and/or graph representing the insertion section).

In accordance with a form of embodiment of the method, the sequence of movements is determined by a trained machine-learning model, where this model has been trained with a plurality of three-dimensional shapes of the insertion section (e.g., in the form of a model and/or graph representing the insertion section, which is linked to a specified sequence of movements as a basic truth).

In one form of embodiment of the method, the sequence of movements contains one or more of the following movements: turning the hand section around the fiber on the hand section as an axis of rotation (e.g., against a torsional force applied to the hand section); retracting the hand section (e.g., in order to remove the tip of the fiber from a critical region); moving the hand section orthogonally to the fiber (e.g., in order to achieve a new attachment point for rotations or for pulling).

In this case, complex movements may be specified (e.g., those that are similar to the movements for untying a knot).

In the case of an apparatus of the present embodiments, the optical fiber is arranged in or on a medical instrument (e.g., a catheter, an endoscope, or a guide wire or tube).

An apparatus of the present embodiments includes a movement unit (e.g., a surgical robot) configured to move the holding section of the fiber automatically according to the sequence of movements (e.g., configured to process a command sequence generated from the sequence of movements).

For the creation of the sequence of movements, AI-based methods (AI: “Artificial Intelligence”) may be used for the method of the present embodiments. Artificial intelligence is based on the principle of machine-based learning and is generally performed with an adaptive algorithm that has been trained accordingly. The term “machine learning” is often used for machine-based learning, the principle of “deep learning” also being included here.

Components of the present embodiments (e.g., the movement determination unit) may be present as a “cloud service”. Such a cloud service serves to process data (e.g., using artificial intelligence) but may also be a service based on conventional algorithms or a service in which an evaluation by humans takes place in the background. In general, a cloud service (also referred to below as the “cloud”) is an IT infrastructure in which, for example, storage space or computing power and/or application software is made available via a network. The communication between the user and the cloud takes place using data interfaces and/or data transmission protocols. In the present case, the cloud service may provide both computing power and application software.

In connection with a method of the present embodiments, a provision of data obtained in connection with the present embodiments takes place to the cloud service via the network. This includes a computing system that may not include the user's local computer. The method may be implemented using a command constellation in a network. The data calculated in the cloud is later sent back to the user's local computer via the network.

### BRIEF DESCRIPTION OF THE DRAWINGS

The present embodiments are described in greater detail below with reference to the appended figures using example embodiments. In this case, the same components are provided with same reference characters in the various figures. The figures are generally not to scale.

FIG. 1 shows an embodiment of an apparatus for controlling movement of an elongated medical instrument within a body;

FIG. 2 shows an embodiment of a method for controlling movement of an elongated medical instrument within a body; and

FIG. 3 shows a relaxation of a fiber.

### DETAILED DESCRIPTION

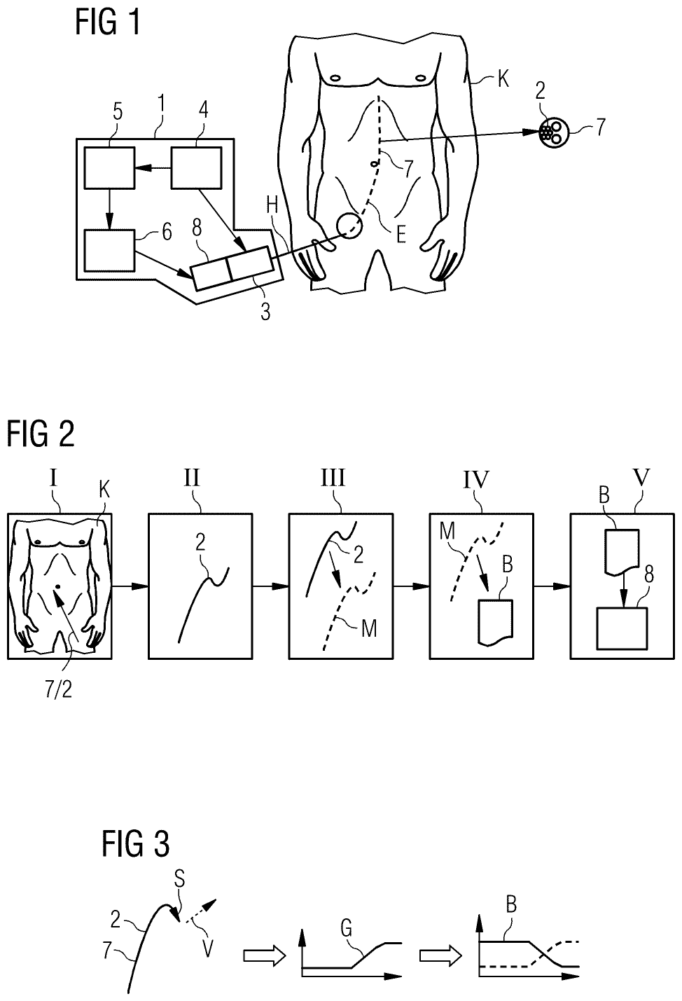

FIG. 1 shows an apparatus 1 for controlling movement of an elongated medical instrument 7 (e.g., an instrument) within a body K. The instrument (e.g., a catheter or a guide tube) has been inserted through an incision (e.g., see circle on the groin) into a body K and slid upward in the direction of the heart (e.g., through a blood vessel). A cross-section through the instrument is shown on the right. Inside the instrument, elements (individual circles) (e.g., cables or wires) for control or imaging and an optical fiber 2 configured for optical shape capture run along an entire length of the instrument 7. This fiber 2, for example, consists of seven optical waveguides that are combined to form a bundle.

The part of the fiber 2, and thus also of the instrument 7, located in the body K is the insertion section E. The instrument may be guided at a holding section H.

An example of an apparatus 1 of the present embodiments is shown on the left. The apparatus 1 includes a measuring unit 3, a movement determination unit 5, a data interface 6, and a movement unit 8.

The measuring unit 3 is configured to measure a bend at multiple positions of the fiber 2. The shape determination unit 4 is configured to determine the three-dimensional shape of the insertion section E in the body K from the measured bend. In this example, the shape determination unit 4 is also configured to determine the positions and directions of the bending forces prevailing there. The one movement determination unit 5 is configured to determine a sequence of movements B. This sequence of movements B is based on the shape of the insertion section E and the bending forces prevailing there. If the holding section H of the instrument 7 is moved with this sequence of movements B, the bend of the instrument 7 in the insertion section E may be decreased if the holding section H is moved correspondingly.

The sequence of movements B may be output via a data interface 6. In this case, the sequence of movements B is composed of control commands for the automated control of the movement unit 8. This movement unit 8 may, for example, be a surgical robot and is configured to move the holding section H of the fiber 2 automatically in accordance with the sequence of movements B.

In this example, the instrument 7 is already optimally inserted into the body K. The function of the apparatus is described more precisely below using examples.

FIG. 2 shows a method for controlling the movement of an elongated medical instrument 7 within a body K.

In act I, the instrument 7 is slid into the body K, where the optical fiber 2 on the instrument 7 is slid into the body K via an insertion section E.

In act II, the bend of the fiber 2 in its insertion section E is measured at multiple positions, and the three-dimensional shape of the insertion section E of the fiber 2 in the body K is determined from the measured bend and, where appropriate, also the positions and directions of the bending forces prevailing there.

In act III, a model of the fiber 2 is created from the three-dimensional shape of the insertion section E of the fiber 2 in the body K.

In act IV, a sequence of movements B is determined based on the shape of the insertion section E using the model M. With this sequence of movements B, a holding section H of the instrument 7 is to be moved, so that the bending forces of the instrument 7 in the insertion section E may be decreased if the holding section H is moved correspondingly.

The sequence of movements B is then output in act V to a movement unit of the apparatus in accordance with FIG. 1.

FIG. 3 outlines the procedure of relaxation of a fiber 2. On the left, the instrument 7 and thus also the fiber 2 has bent while being slid in (e.g., in a direction of the arrow) in the area of the tip S. Due to the restoring torque of the instrument, a force vector V now acts there, which is opposed to the bend. After a measurement of the fiber 2, a graph G of the bending forces is created. Using this graph (e.g., indicated by dots on the right), the sequence of movements B is then created.

In conclusion, it is once again noted that the invention described in detail above relates solely to example embodiments that may be modified by the person skilled in the art in a variety of ways, without departing from the scope of the invention. Further, the use of the indefinite article “a” or “an” does not rule out that the features in question may also be present multiple times. Likewise, terms such as “unit” do not rule out that the components in question consist of multiple interacting subcomponents that, where appropriate, may also be distributed spatially. The term “a number of” is to be understood as “at least one.” Independent of the grammatical term usage, individuals with male, female, or other gender identities are included within the term.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for controlling movement of an elongated medical instrument within a body that, along a length of the elongated medical instrument, includes an optical fiber configured for optical shape capture, wherein an insertion section of the optical fiber is located in the body, the method comprising:
measuring a bend at multiple positions of the optical fiber, at least along the insertion section of the optical fiber;
determining a three-dimensional (3D) shape of the insertion section of the optical fiber in the body from the measured bend;
determining a sequence of movements based on the 3D shape of the insertion section, with which a holding section of the elongated medical instrument is to be moved, so that the bend of the elongated medical instrument in the insertion section is decreasable when the holding section is moved correspondingly; and
outputting the sequence of movements.

2. The method of claim 1, further comprising determining positions and directions of bending forces prevailing at the bend.

3. The method of claim 2, wherein the determined sequence of movements is also based on the bending forces prevailing at the bend.

4. The method of claim 1, wherein determining the 3D shape of the insertion section of the optical fiber comprises creating a 3D model of the insertion section, a graph that shows bending information along the length of the insertion section, or a combination thereof.

5. The method of claim 4, wherein the determined sequence of movements is also based on the 3D model, the graph, or the 3D model and the graph.

6. The method of claim 2, wherein determining the bending forces comprises calculating force vectors along the length of the insertion section,
wherein the force vectors are classified into a 3D model of the insertion section, are classified into a graph that shows the bending information along the length of the insertion section, or classified into the 3D model of the insertion section and classified into the graph that shows the bending information along the length of the insertion section.

7. The method of claim 1, wherein while the elongated medical instrument is being slid in, the bend is determined multiple times.

8. The method of claim 7, wherein the 3D shape of the insertion section of the optical fiber is determined at intervals of time of less than one second or less than 0.1 s until the insertion of the optical fiber has stopped.

9. The method of claim 1, wherein the sequence of movements comprises a successive sequence of movements of the holding section that specifies how the holding section has to be moved, so that the bending forces in the insertion section are reduced.

10. The method of claim 1, further comprising:
moving the holding section of the optical fiber in accordance with the sequence of movements;
displaying the sequence of movements for performance by a human;
creating a command sequence from the sequence of movements configured so that an automatic movement unit is configured to move the holding section automatically in accordance with the sequence of movements; or
any combination thereof.

11. The method of claim 10, wherein the outputting of the sequence of movements, the moving, the displaying, the creating, or any combination thereof is started automatically when one or more of the following conditions apply:
a bending radius is smaller than a specified limit value;
a bending force lies above a specified limit value;
a number of bends in a specified length interval of the insertion section lies above a specified limit value;
a sum of the bending forces in a specified length interval of the insertion section lies above a specified limit value; and
parameter values of a shape of a model of the insertion section or of a shape of the insertion section shown in an image lie outside a specified value range.

12. The method of claim 2, wherein determining the sequence of movements comprises determining the sequence of movements analytically from an analysis of the determined 3D shape of the insertion section and the positions and directions of the bending forces prevailing at the bend.

13. The method of claim 12, wherein determining the sequence of movements analytically from the analysis of the determined 3D shape of the insertion section and the positions and directions of the bending forces prevailing at the bend comprises analyzing a model, a graph, or the model and the graph representing the insertion section.

14. The method of claim 1, wherein the sequence of movements is determined by a trained machine-learning model, and
wherein the trained machine-learning model has been trained with a plurality of 3D shapes of the insertion section in the form of a model, a graph, or the model and the graph representing the insertion section, which were linked to a specified sequence of movements as a basic truth.

15. The method of claim 1, wherein the sequence of movements contains one or more of the following movements:
turning a hand section around the optical fiber on a hand section as an axis of rotation, against a torsional force applied to the hand section;
retracting the hand section in order to remove a tip of the optical fiber from a critical region; and
moving the hand section orthogonally to the optical fiber in order to achieve a new attachment point for rotations or for pulling.

16. An apparatus for controlling movement of an elongated medical instrument within a body, the elongated medical instrument including, along a length of the body, an optical fiber configured for optical shape capture, wherein an insertion section of the optical fiber is located in the body, the apparatus comprising:
a measuring unit configured to measure a bend at multiple positions of the optical fiber, at least along the insertion section of the optical fiber;
a shape determination unit configured to determine the three-dimensional (3D) shape of the insertion section in the body from the measured bend;
a movement determination unit configured to determine a sequence of movements based on the 3D shape of the insertion section, with which a holding section of the elongated medical instrument is to be moved, so that the bend of the elongated medical instrument in the insertion section is decreasable when the holding section is moved correspondingly; and
a data interface configured to output the sequence of movements.

17. The apparatus of claim 16, wherein the optical fiber is arranged in or on a medical instrument, an endoscope, or a guide wire or tube.

18. The apparatus of claim 16, further comprising a movement unit configured to move the holding section of the optical fiber automatically in accordance with the sequence of movements.

19. The apparatus of claim 18, wherein the movement unit is further configured to process a command sequence generated from the sequence of movements.

20. In a non-transitory computer-readable storage medium that stores instructions executable by one or more processors to control movement of an elongated medical instrument within a body that, along a length of the elongated medical instrument, includes an optical fiber configured for optical shape capture, wherein an insertion section of the optical fiber is located in the body, the instructions comprising:
measuring a bend at multiple positions of the optical fiber, at least along the insertion section of the optical fiber;
determining a three-dimensional (3D) shape of the insertion section of the optical fiber in the body from the measured bend;
determining a sequence of movements based on the 3D shape of the insertion section, with which a holding section of the elongated medical instrument is to be moved, so that the bend of the elongated medical instrument in the insertion section is decreasable when the holding section is moved correspondingly; and
outputting the sequence of movements,
wherein the optical fiber is a multifunctional shape-sensing fiber, and a movement of the optical fiber corresponds to an output of corresponding control commands.
